(19) United States
(12) Patent Application Publication
(10) Pub. No.: US 2026/0284427 A1
(43) Pub. Date: Sep. 24, 2026

(54) INTEGRATED, NONINVASIVE STIMULATION DELIVERY SYSTEM AND METHOD FOR TREATING NEURODEGENERATIVE DISEASE SYMPTOMS

(71) Applicant: Andrea Byers, Bristow, OK (US)

(72) Inventor: Andrea Byers, Bristow, OK (US)

(21) Appl. No.: 19/660,992

(22) Filed: Apr. 28, 2026

Related U.S. Application Data
(63) Continuation-in-part of application No. 18/319,082, filed on May 17, 2023, now Pat. No. 12,636,515.
(60) Provisional application No. 63/342,700, filed on May 17, 2022.

(51) Int. Cl. A61N 5/06; A61H 9/00; A61H 21/00; A61H 23/02; A61N 5/067

(52) U.S. Cl. CPC A61N 5/0622; A61H 9/0092; A61H 21/00; A61H 23/0254; A61N 5/0603; A61N 5/067; A61H 2201/0103; A61H 2201/0188; A61H 2201/10; A61H 2201/1238; A61H 2201/165; A61H 2205/02; A61H 2205/04; A61H 2205/08; A61H 2209/00; A61N 2005/0606; A61N 2005/0651; A61N 2005/0659; A61N 2005/0663

(57) ABSTRACT

Disclosed is a vasopneumatic photobiomodulation (PBM) vest that extends the treatment system by adding thoracic and abdominal coverage to the head/neck/face transcranial garment and intraoral device already claimed. The vest integrates: (1) five-cell sequential vasopneumatic compression targeting the thoracic lymphatic network; (2) a tri-wavelength LED and laser diode array (660 nm, 850 nm, and 1064 nm) delivering photobiomodulation across the chest and abdomen; and (3) a rechargeable lithium polymer battery system providing approximately three hours of continuous operation. The vest is designed to complement and extend the glymphatic/lymphatic drainage system disclosed in the parent application, treating the same neurodegenerative disease indications, particularly Alzheimer's disease.

### CROSS-REFERENCE TO RELATED APPLICATIONS

This application claims priority to and is a continuation-in-part of U.S. patent application Ser. No. 18/319,082 filed May 17, 2023, which claims the benefit of U.S. Provisional Application No. 63/342,700, filed on May 17, 2022, the disclosures of which are herein incorporated by reference as if fully set forth herein in their entireties.

### BACKGROUND OF THE INVENTION

### 1. Field of the Invention

The invention disclosed herein is directed to an integrated, noninvasive stimulation delivery system and method for treating neurodegenerative disease symptoms. The inventive system and method integrate noninvasive stimulation delivery technologies, e.g., vasopneumatic compression, photobiomodulation, and vibration, to synergistically stimulate glymphatic flow to consistently clear toxins from a patient's brain with mild-to-moderate dementia and other neurodegenerative disease symptoms.

### 2. Description of the Related Art

Aging is a major risk factor for many neurological disorders, including Alzheimer's disease (AD). Although Alzheimer's is not normal aging, age is the biggest risk factor for the disease. According to the Alzheimer's Association of America, the U.S. population aged 65 and over is expected to double by 2030. Due to projected increases in AD diagnoses in the U.S., the estimated cost to third-party pay systems will increase to $20 trillion over the next 40 years. Another primary concern for these costs is that Alzheimer's makes treating other diseases more expensive, as most individuals with AD have one or more comorbidities that complicate the management of the condition and increase costs.

With Alzheimer's, it isn't just those with the disease who endure the devastating consequences of the disease with declining memory, cognition, and reduced quality of life. AD also significantly impacts the caregivers and families. Caring for an individual with Alzheimer's takes longer, lasts longer, is more personal and invasive, and takes a heavy toll on the caregiver's health. More than 60% of Alzheimer's caregivers rate the emotional stress of caregiving as high or very high, with one-third reporting symptoms of depression. Consequently, caregivers may have negative impacts on their own health, employment, income, and family finances, considering the physical and emotional toll of caregiving.

The mechanisms behind brain waste clearance from the parenchyma through meninges and vessels with molecular markers (e.g., endothelial cells of conventional lymphatic vessels) play a paramount role in cerebrospinal fluid (CSF) drainage. These mechanisms can successfully clear macromolecules and immune cells from the subarachnoid space into the cervical lymph nodes. Since these vessels do not reach the parenchyma, complementary mechanisms are required. In that regard, the brain has another clearance mechanism in which interstitial solute transport across the blood-brain barrier (BBB) drains into the bloodstream. However, this route can be hindered by the large distances between interstitial solutes and the BBB. Moreover, the tightly sealed endothelium of brain capillaries precludes normal systemic interstitial and lymphatic flow into the brain. To bypass this situation, other clearance routes are favored by the body, such as CSF-interstitial fluid (ISF) bulk flow of the glymphatic system.

A breakdown in CSF-ISF exchange occurs in Alzheimer's disease and other neurological conditions. Alzheimer's is the most common type (60-70% of total) of dementia and is mainly characterized by hyperphosphorylated tau and amyloid-β (Aβ) protein deposition, wherein an imbalance between Aβ production and clearance results in toxic accumulation (FIG. 1). One factor influencing CSF movement is arterial pulsatility and movement of vessel walls caused by the cardiac cycle. Arteries, when pulsating, momentarily increase pressure on the surrounding fluid, extravasating the perivascular space. The positive pressure of CSF production drives its movement from the choroid plexus and is supported by the activity of cilia and the process of deep respiration. Astrocytes allow fluid movement between perivascular spaces and the interstitium via water channels (AQP4, FIG. 2).

The pathway for CSF fluid drainage occurring between the arachnoid and the pia matter of the meninges can drain into the bloodstream through the arachnoid granulations located along the superior sagittal sinus and the transverse sinuses. Alternatively, CSF fluid drainage can reach the lymphatic vasculature of the nasal mucosa by crossing the cribriform plate, localized under the olfactory bulbs, along the olfactory nerves. The meningeal lymphatics enable the drainage of macromolecules and immune cells (FIG. 2). While the CSF fluid drains back to the bloodstream, the macromolecules and immune cells localized within the CSF drain primarily through the meningeal lymphatic vessels to reach the deep cervical lymph nodes.

The glymphatic system is a concept associated with CSF and ISF dynamics within the CNS (FIG. 3). The term glymphatic is derived from glial and lymphatic systems. The glymphatic system does not represent the discovery of a previously unknown anatomical structure but rather a brain-wide paravascular pathway from CSF and ISF exchange that facilitates the efficient clearance of macromolecules and waste from the brain (FIG. 3). In AD, Aβ congestion is both a symptom and disease indicator. Specifically, individuals with AD have impaired perivascular circulation, which inhibits Aβ from readily being removed. In addition, aging has been associated with peripheral lymphatic dysfunction affecting T cell egress and macromolecule drainage. In peripheral tissues, circulating ISF is recycled into the local lymphatic system, thereby cleaning tissues and preventing the accumulation of potentially toxic compounds, such as cellular metabolites or debris from damaged or necrotic cells.

A major risk factor for many neurological pathologies, such as Alzheimer's, poses a global health challenge associated with declining memory, cognition, and reduced quality of life. Current medications and treatments for treating Alzheimer's disease symptoms display notable side effects, including nausea, vomiting, constipation, dizziness, impaired alertness, impaired motor coordination, worsening depression, suicide ideation, sleep paralysis, and compromised respiratory function. Recent findings show that meningeal and cranial nerve glymphatic decongestion may affect the removal of the tau and Aβ protein plaques from the central nervous system (CNS). The glymphatic system, i.e., aquaporin 4 (AQP4)-facilitated exchange of cerebrospinal fluid (CSF) with interstitial fluid (ISF), may provide a promising clearance pathway for these diseases; however, a significant obstacle to treating these diseases is connecting the new glymphatic flow physiological science to affordable, applicable daily modalities for individuals with Alzheimer's disease. These modalities effectively remove glymphatic congestions, such as accumulated amyloid proteins, a major contributing factor to the progression of this neurological condition.

Currently, studies are underway to test techniques and effectiveness in clearing Aβ and tau from the central nervous system. One such technique is photobiomodulation (PBM), which is a dual-modality method for treating neurodegenerative diseases. PBM is gaining attention as a safe, antiviral, and anti-inflammatory approach to treat neurodegeneration with photons, stimulate mitochondrial increases in ATP and proteasomes, and aid in misfolded protein removal. PBM alters multiple genes' expression and upregulates multiple pathogenic pathway inflammation, reactive oxidative stress, mitochondrial disorders, insulin resistance, epigenetic defects, regulation of neuroprotective factors, and regional hypoperfusion in the brain.

Only minimal manual neurolymphatic drainage has been attempted by trained and certified skilled therapists that can provide such glymphatic and lymphatic decongestion. These skilled individuals can be costly, and their availability is sparse. Therefore, it is desirable to provide an improved system and method for treating Alzheimer's disease that will decrease the burden on healthcare systems and caregivers while also improving daily life for sufferers of cognitive impairments, including alleviating dependence on others.

Before proceeding to a brief summary and subsequent detailed description of the invention, however, it should be noted and remembered that the description of the invention which follows, together with the accompanying drawings, should not be construed as limiting the invention to the examples (or embodiments) shown and described. Those skilled in the art to which the invention pertains will be able to devise other forms of this invention within the ambit of the appended claims.

### BRIEF SUMMARY OF THE INVENTION

The present disclosure relates to integrated, noninvasive stimulation delivery systems and methods configured to facilitate lymphatic and glymphatic fluid movement within a user. In particular embodiments, the disclosed systems apply combinations of vasopneumatic compression, photobiomodulation, and vibration to anatomical regions associated with cranial, cervical, thoracic, and abdominal lymphatic drainage pathways. More particularly, the invention disclosed herein provides an integrated, noninvasive stimulation delivery system and method for use in associate with neurological conditions, namely treating Alzheimer's disease, and more particularly, a system and method that integrate noninvasive stimulation delivery technologies, e.g., vasopneumatic compression, photobiomodulation, and vibration, to synergistically stimulate glymphatic flow to consistently clear toxins from a patient's brain and body with mild-to-moderate dementia and other neurodegenerative disease symptoms.

It is further desirable to provide a system and method that sequentially integrate photobiomodulation with vibration in an intraoral device to stimulate glymphatic pathway clearance of toxins toward the cranial nerve pathway and into the systemic lymphatic pathway and with a vasopneumatic transcranial device to stimulate lymphatic flow at the cranial nerve exit of the skull in order to decrease tau and Aβ, consequently increasing cognition and independence, in daily living activities, in individuals with Alzheimer's disease. In certain embodiments, a wearable vest is provided as an optional component that may be used alone or in combination with the transcranial and/or intraoral devices. The vest is configured to apply vasopneumatic compression and photobiomodulation to thoracic and abdominal regions so as to facilitate lymphatic fluid propulsion toward central lymphatic outflow pathways. Together, the intraoral device, the transcranial device, and the vest create a coordinated pressure and photobiomodulation treatment along the full lymphatic drainage pathway.

In general, the invention enhances glymphatic decongestion and lymphatic efflux by combining photobiomodulation, vibration techniques, and vasopneumatic compression in an intraoral device, a transcranial device, and a vest. The transcranial device utilizes vasopneumatic compression and photobiomodulation to stimulate cervical lymphatic flow and transcranial lymphatic flow. The vest utilizes vasopneumatic compression to propel lymph towards the left subclavian vein and photobiomodulation to aid in glymphatic clearance of amyloid-beta. The three-device system is used to facilitate clearance pathways associated with protein transport from the CNS. The transcranial device opens a superficial pathway for removing toxins and proteins from perivascular spaces to maintain the homeostasis of protein and lymph through transcranial lymphatic channels, while the vest provides thoracic and abdominal coverage. The inventive system and method apply vasopneumatic transcranial pressures to route lymphatic fluid toward the subclavian lymphatic pathway for effective and efficient lymphatic congestion clearing as well as thoracic and abdominal vasopneumatic pressure to route lymphatic fluid towards the left subclavian vein.

The intraoral device eliminates the need for a trained health care provider to administer the intraoral protocol while ensuring continuous treatment delivery through vibration and photobiomodulation of seventeen stimulation points on the hard and soft palate. The intracranial device decongests brain tau and amyloid proteins and toxins towards the superficial lymphatic pathway through the cranial nerves. Perivascular lymphatic drainage from the glymphatic and meningeal pathways prohibits the build up of tau and amyloid peptides, maintains homeostasis of the cerebrospinal fluid, and averts cognitive decline in Alzheimer's disease. The glymphatic network serves as the initial waste clearance for proteins and metabolic products via connection to the authentic lymphatic network associated with the dura covering of the brain and cranial nerves and large vessels at the skull exits. Virchow-Robin Spaces and the Arachnoidal Septi, along the olfactory and optical nerves, transport intracranial lymphatic load towards the subclavian lymph nodes through cervical lymph pathways; this flow is enhanced through manual intraoral drainage procedures.

Removal of hyperphosphorylated tau and amyloid-beta from the brain improves cognition and functional activities of daily living, as well as overall independence. The noninvasive devices disclosed herein stimulate glymphatic flow, have shown no side effects, and are easily tolerated in everyday use. Stimulating glymphatic and transcranial flow enhances protein clearance from the CNS to benefit daily functional outcomes.

The invention disclosed herein provides an innovative treatment modality by combining a transcranial device and an intraoral device to remove toxins from the CNS. These devices remove hyperphosphorylated tau and amyloid beta from brains of individuals over age 65, with mild-to-moderate cognitive impairment, evidenced by baseline blood work, using blood testing for hyperphosphorylated tau, amyloid beta-40, (Aβ40, Aβ42), Apolipoprotein E prototype, Amyloid Probability Score (APS, calculated from plasma Aβ42/40 ratios), and participant age. This is coupled with the Saint Louis Mental Status Examination (SLUMS) score at baseline and at 90 days. Daily treatments with these two devices stimulate the removal of Aβ from the CNS.

The foregoing has outlined in broad terms some of the more important features of the invention disclosed herein so that the detailed description that follows may be more clearly understood. The invention is not limited in its application to the details of the construction and to the arrangements of the components set forth in the following description or illustrated in the drawings. Instead, the invention is capable of other embodiments and of being practiced and carried out in various other ways not specifically enumerated herein. Finally, it should be understood that the phraseology and terminology employed herein are for the purpose of description and should not be regarded as limiting unless the specification specifically so limits the invention.

### BRIEF DESCRIPTION OF THE DRAWINGS

These and further aspects of the invention are described in detail in the following examples and accompanying drawings.

FIG. 1 illustrates amyloid plaque formation and brain pathology in Alzheimer's disease.

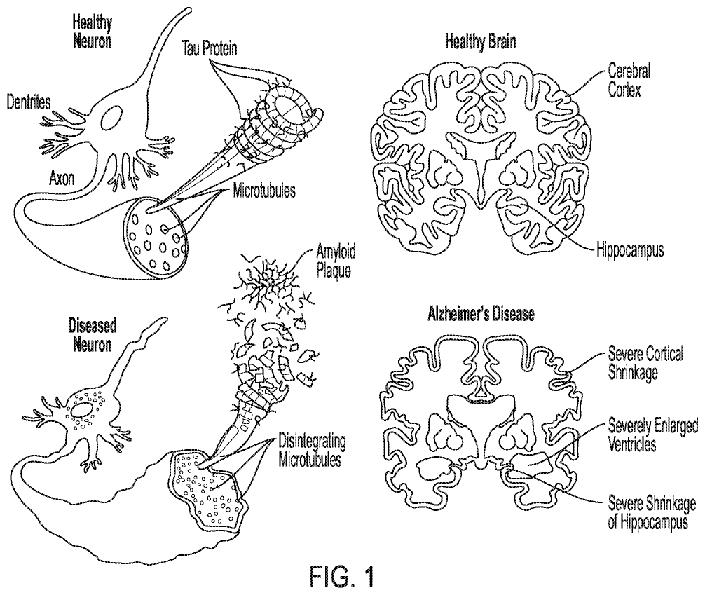

FIG. 2 illustrates the glymphatic system.

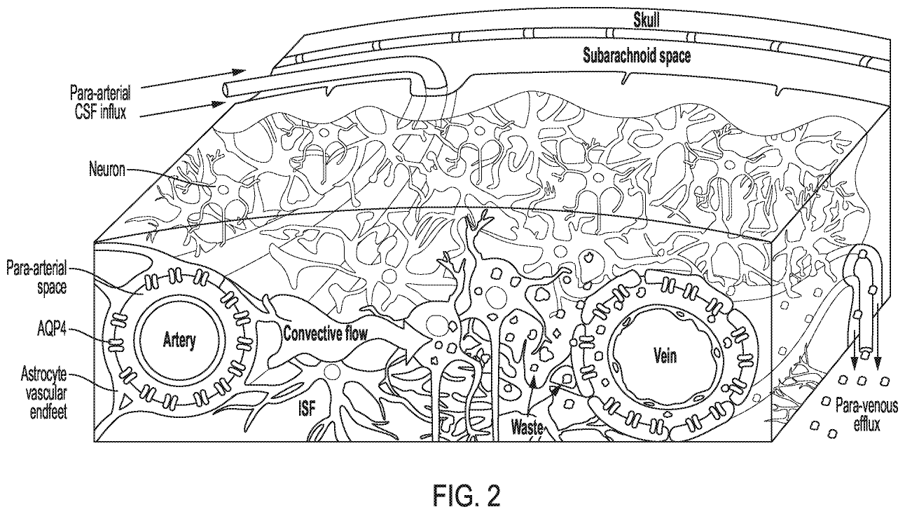

FIG. 3 illustrates the clearance of Aβ by degradation and absorption into the cerebrospinal fluid and interstitial fluid to/from the bloodstream.

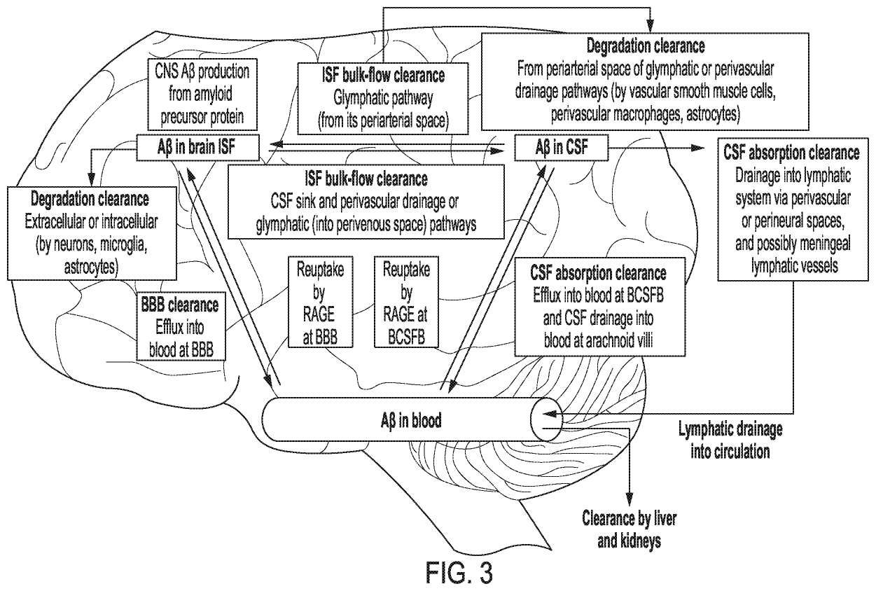

FIG. 4 is an exploded, perspective view of an example of a transcranial sequential vasopneumatic device configured to pulse 810 nm near-infrared light in accordance with an illustrative embodiment of the invention disclosed herein.

FIG. 5 is an electrical schematic of an example of a transcranial sequential vasopneumatic device in accordance with an illustrative embodiment of the invention disclosed herein.

FIG. 6 is an exploded, perspective view of an example of an intraoral device configured to emit 670 nm near-infrared light and provide vibration stimulation to seventeen (17) points in the hard and soft palate in accordance with an illustrative embodiment of the invention disclosed herein.

FIG. 7 is an electrical schematic of an example of an intraoral device in accordance with an illustrative embodiment of the invention disclosed herein.

FIG. 8 is a front, elevation view of an example of a vasopneumatic photobiomodulation vest in accordance with an illustrative embodiment of the invention disclosed herein.

FIG. 9 is a rear, elevation view of the vasopneumatic photobiomodulation vest shown in FIG. 8.

FIG. 10 is cross-sectional view of a portion of the vasopneumatic photobiomodulation vest shown in FIG. 8.

FIG. 11 is an exploded, cross-sectional view of a portion of the vasopneumatic photobiomodulation vest shown in FIG. 8.

FIG. 12 is a block diagram of an example of a vasopneumatic photobiomodulation vest in accordance with an illustrative embodiment of the invention disclosed herein.

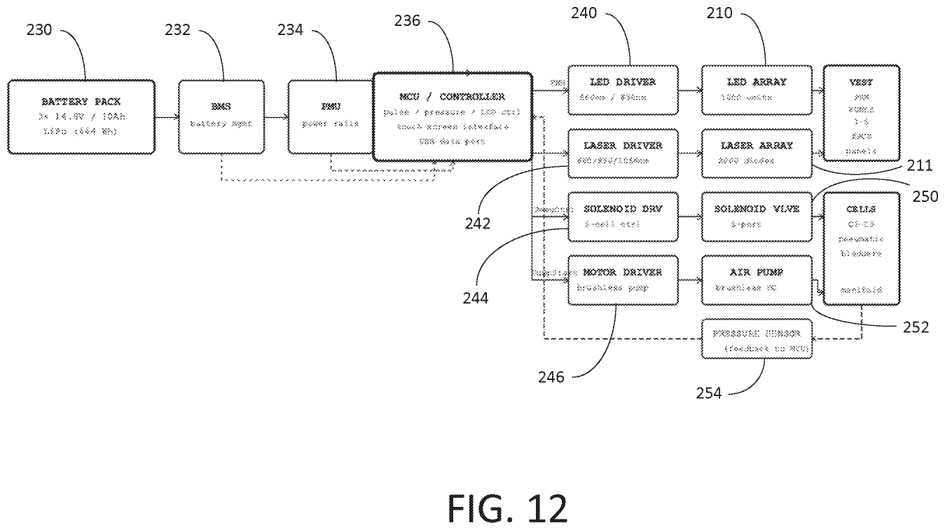

FIG. 13 is a cross-sectional view of a battery module in accordance with an illustrative embodiment of the invention disclosed herein.

### DETAILED DESCRIPTION OF THE INVENTION

While this invention is susceptible of embodiment in different forms, there are shown in the drawings and will herein be described hereinafter in detail some specific embodiments of the invention. However, it should be understood that the present disclosure is to be considered an exemplification of the principles of the invention and is not intended to limit the invention to the specific embodiments so described.

The invention is concerned with an innovative treatment strategy that involves the synergistic use of a transcranial device and an intraoral device, specifically designed to aid in the clearance of macromolecules, including but not limited to amyloid-beta (AB) and tau proteins, from the Central Nervous System (CNS) in individuals experiencing mild-to-moderate cognitive impairment as a result of Alzheimer's disease. This unique approach seeks to alleviate glymphatic congestion by stimulating meningeal decongestion, effectively rerouting these macromolecules towards cranial nerve lymphatic pathways for subsequent removal from the CNS.

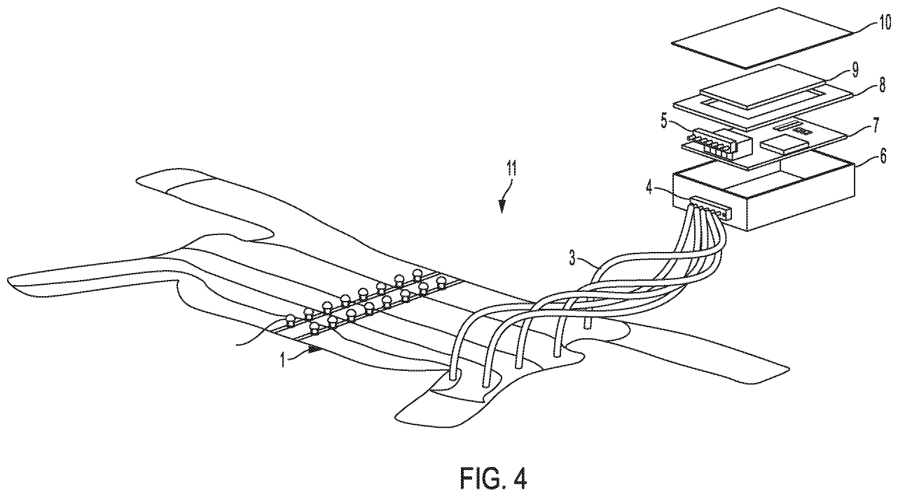

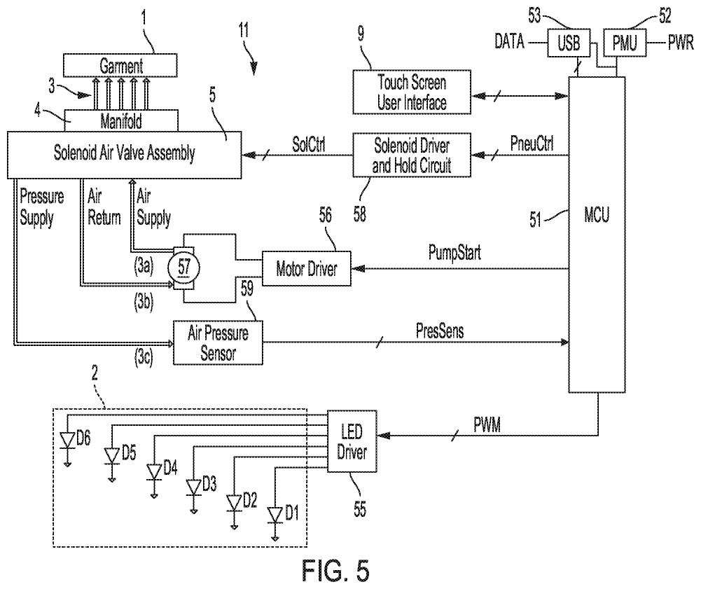

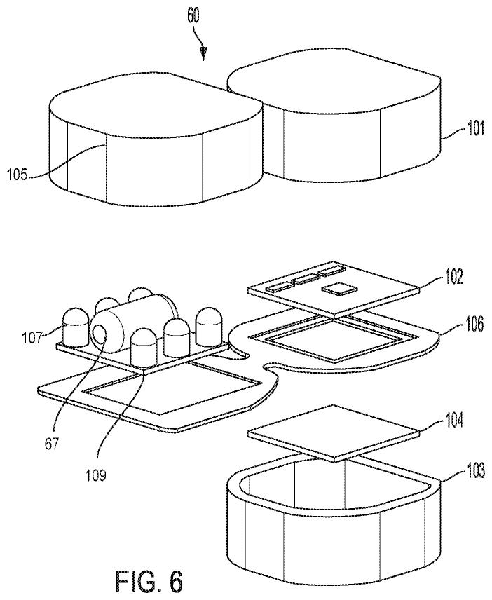

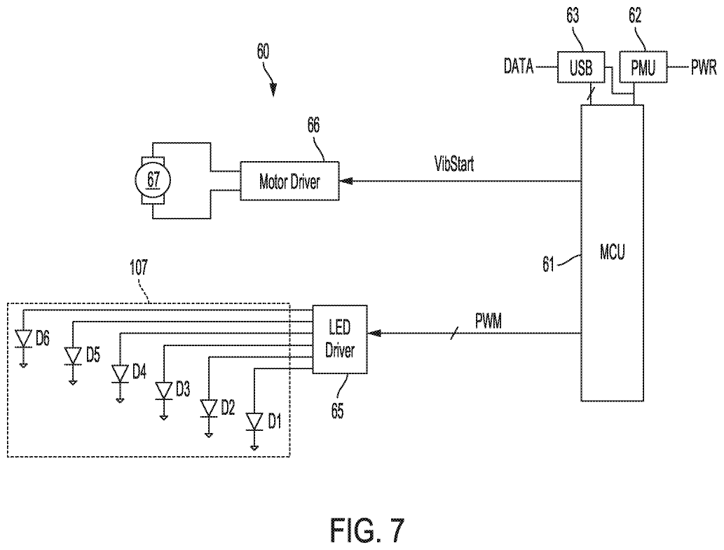

The system and method detailed herein aims to enhance glymphatic and lymphatic flow within the CNS through the application of photobiomodulation, which is achieved using a transcranial vasopneumatic device 11 (as depicted in FIGS. 4 and 5) and a 670 nm LED Near-Infrared (NIR) intraoral device 60 (as illustrated in FIGS. 6 and 7). This system is further augmented by the inclusion of vibration stimulation targeting 17 specific points identified within the hard and soft palates of each individual patient.

The transcranial device 11, depicted in FIGS. 4 and 5, features a five-cell sequential vasopneumatic compression garment 1, designed for application on the head, neck, and face. To ensure comfort and suitability, the garment 1 is produced in a range of sizes to accommodate varying head and neck circumferences of individual users.

The garment 1 is outfitted with outlets that are fluidly connected to multiple air tubes 3. These tubes, in turn, are fluidly linked to an air tube bulkhead connector or manifold 4, mechanically attached to a housing 6. The manifold 4 is fluidly connected to a solenoid valve assembly 5 housed within the housing 6. The solenoid valve assembly 5 operates under control signals SolCtrl generated by a solenoid driver 58. The solenoid valve assembly 5 is fluidly linked to an air pump 57 (such as a brushless DC motor pump) operated by a motor driver 56. Both these components are situated within the housing 6.

Further detailing these connections: an air supply line 3a fluidly links the air pump 57 to the solenoid valve assembly 5, facilitating air flow from the pump 57 to the solenoid valve assembly 5. A return air supply line 3b fluidly connects the solenoid valve assembly 5 to the air pump 57. A pressure supply line 3c fluidly links the solenoid valve assembly 5 to an air pressure sensor 59 located within the housing 6. This sensor is vital for detecting the air pressure within the manifold 4. Together, these components orchestrate the vasopneumatic compression on the user.

The garment 1 also integrates an array of 810 nm pulsed NIR LEDs 2. These LEDs serve as the primary source of photobiomodulation and are controlled by an LED driver 55.

Within the housing 6 of the transcranial device 11, there is a printed circuit board (PCB) assembly 7. This is powered by a power management unit (PMU) 52, which receives power from a suitable source, such as a lithium-ion battery. The PCB assembly 7 houses a microcontroller 51, which functions as a Human-Machine Interface (HMI) controller. The microcontroller 51 features a USB interface, allowing data transmission via a USB port 53 with an external device. This interface provides a path for programming the microcontroller 51 or collecting data from it.

The transcranial device 11 also includes a touch screen 9, which is mounted on a support or holder 8 on the housing 6, and shielded by a touch screen cover 10 (typically transparent glass). The touch screen 9 is electrically connected to the microcontroller 51 and, under the control of the microcontroller 51, displays operational options to the user and accepts input commands.

Upon receiving user input via the touch screen 9, the microcontroller 51 generates a pneumatic control signal (PneuCtrl). This PneuCtrl instructs the solenoid driver 58 to generate the appropriate solenoid control signals (SolCtrl). It also produces a pump start signal (PumpStart) that directs the motor driver 56 to operate the air pump 57 suitably, executing vasopneumatic compression on the user.

Simultaneously, the microcontroller 51 generates a pulse width modulation (PWM) signal for the LED driver 55. This signal instructs the LED driver 55 to drive the NIR LEDs 2, resulting in the delivery of suitable red light pulsing for the photobiomodulation therapy applied to the user. This integrated functionality helps the transcranial device 11 attempt to provide a comprehensive and coordinated treatment, with the aim of addressing the cognitive impairment in Alzheimer's disease patients.

Referring now to FIGS. 6 and 7, the intraoral device 60, designed for stimulating glymphatic decongestion, incorporates a molded mouthpiece assembly 105, which is securely attached to upper 101 and lower 103 external assembly covers. The mouthpiece assembly 105 is engineered to adapt to a broad spectrum of variations in individuals' hard palate structures. The design of the intraoral device 60 aims to align seventeen stimulation pressure points located in the hard palate accurately against the stimulation face of the mouthpiece.

Nestled within the mouthpiece assembly 105 is a printed circuit board (PCB) 109, which is affixed to a device holder 106. The PCB 109 accommodates a linear vibration motor 67 and a series of 670 nm visible red and Near-Infrared (NIR) LEDs 107. Also situated on the PCB 109 are a motor driver 66, which controls the linear vibration motor 67 in response to a vibration start signal (VibStart), and an LED driver 65, which drives the LEDs 107 according to a received pulse width modulation (PWM) signal. This PWM signal instructs the LED driver 65 to activate the LEDs 107, resulting in the delivery of appropriate red light pulsing for the photobiomodulation therapy administered to the user.

A power management unit (PMU) 62 is also mounted to the PCB 109. It serves to regulate the power supply from the battery to the PCBs 109 and 102.

The device holder 106 extends within the upper 101 and lower 103 external assembly covers. A rechargeable battery 104, for instance, a lithium-ion battery, is positioned within the lower 103 external assembly cover. Simultaneously, a PCB 102 is held by the device holder 106 within the upper 101 external assembly cover. The PCB 102 supports a microcontroller 61, which generates the PWM and VibStart signals.

A USB interface 63 is also mounted on the PCB 102. This interface provides a conduit for data transmission, enabling programming of the microcontroller 61 or data extraction from it.

The inventive system and method will be employed to remove amyloid beta and hyperphosphorylated tau from the brains of individuals over age 65 with mild-to-moderate cognitive impairment, evidenced by baseline blood work, using blood testing for hyperphosphorylated tau, amyloid beta-40, (Aβ40, Aβ42), apolipoprotein E prototype, Amyloid Probability Score (APS, calculated from plasma Aβ42/40 ratios), and participant age. Daily treatments of vibration and photobiomodulation from the intraoral device are combined with transcranial vasopneumatic sequential pressure from the transcranial device 11 to stimulate the removal of Aβ and tau proteins from the CNS via the glymphatic and lymphatic networks. The inventive system and method can be coupled with SLUMS scores of <27 (SLUMS is scored 27-30 with high school education as normal, 21-26, mild neurocognitive disorder, and 0-20 indicating dementia) at baseline, 90 days, and 180 days.

The inventive treatment modality combines transcranial 11 and intraoral 60 devices to remove toxins from the CNS. As a basic example of treatment utilizing the devices, once daily, the patient dons the transcranial device 11 and turns the device on to deliver a sequential preset compression to the head/neck/face and a pulsed light photobiomodulation for a predetermined amount of time (e.g., 30-minute treatment setting). The patient also inserts the intraoral device 60 into their mouth and turns it on to deliver vibration stimulation and photobiomodulation to the hard and soft palates of the user's mouth. These sequential modalities decrease Aβ and Tau proteins and increase cognition for AD patients.

A more detailed treatment is as follows. Treatment is administered once daily, for a duration of 30 minutes per session. The treatment is two-pronged, employing both the transcranial device 11 and the intraoral device 60.

The user begins with the transcranial device 11. Once the device 11 is properly positioned on the user's head, neck, and face, the device 11 is activated. Upon activation, the device 11 delivers a sequence of preset millimeter of mercury (mmHg) compression to the head, neck, and face. This process is accompanied by pulsed light photobiomodulation, all within the span of the 30-minute treatment period. It's noteworthy that the mmHg in the first chamber, also identified as the “superior channel,” is adjustable, and this adjustment decreases by 1 mmHg sequentially across the chambers, from superior to inferior.

As part of the process, the head garment 1 is applied as follows: The head garment 1 is unfolded such that its head strap is facing upwards. The garment 1 is placed around the back of the user's head, aligning the top of the garment 1 so its forehead strap runs across the crown of the head. The forehead strap is then pulled through the plastic loop and tightened by pulling on the strap. It is subsequently secured by attaching the hook fastener of the forehead strap to the head strap. The nose piece is then molded across the bridge of the user's nose, and the free hook fasteners are attached to the side of the head garment to ensure good contact with the user's cheeks. The garment is adjusted as necessary so that the user's vision is not obstructed. The right inner chin strap is adjusted to fit snugly under the user's chin, followed by the left outer chin strap being overlapped over the right inner chin strap and secured with the hook fastener. The garment is designed to be worn without impeding the user's vision, breathing, or swallowing.

Following the application of the transcranial device 11, the user then inserts the intraoral device 60 into their mouth and activates it. This can be performed simultaneously with the transcranial device treatment. The intraoral device 60 delivers vibration stimulation and photobiomodulation to the hard and soft palate of the user.

This dual treatment may effectively encourage glymphatic decongestion and lymphatic flow within the CNS. Indeed, the intraoral device 60 plays a helpful role in the decongestion of hyperphosphorylated tau and amyloid proteins and toxins from the brain. As explained, the device 60 utilizes vibration and photobiomodulation to stimulate seventeen specific points on the hard and soft palate. This activity propels brain hyperphosphorylated tau and amyloid proteins and toxins towards the superficial lymphatic pathway, facilitated by cranial nerves. These actions contribute to the maintenance of cerebrospinal fluid homeostasis, consequently preventing the buildup of harmful hyperphosphorylated tau and amyloid peptides, which are often implicated in the cognitive decline associated with neurodegenerative disorders.

The mechanism underlying this process involves the glymphatic network, which serves as the primary waste clearance system for proteins and metabolic byproducts in the brain. This network communicates with the authentic lymphatic network associated with the dura covering of the brain, as well as cranial nerves and large vessels at the exits of the skull. The Virchow-Robin Spaces and the Arachnoidal Septi, located along the olfactory and optical nerves, further facilitate the transportation of intracranial lymphatic load towards the subclavian lymph nodes through cervical lymph pathways.

Complementing the intraoral device 60, the transcranial device 11 applies vasopneumatic compression and Photobiomodulation to stimulate cervical and transcranial lymphatic flow. This activity routes lymphatic fluid towards the subclavian lymphatic pathway, thus clearing lymphatic congestion effectively and efficiently. The process helps maintain the homeostasis of protein and lymph, ensuring toxins and proteins are removed from perivascular spaces, and supporting overall brain health.

Together, the intraoral 60 and transcranial 11 devices function synergistically to enhance the removal of toxins, including hyperphosphorylated tau and amyloid beta, from the central nervous system (CNS). The stimulation of glymphatic and transcranial flow may enhance protein clearance from the CNS, thereby benefitting the functional daily outcomes of individuals, especially those suffering from neurodegenerative conditions.

In addition to the transcranial device 11 and intraoral device 60, a vest 200 can be used alone, with the transcranial device 11, with the intraoral device 60, or any combination thereof. The vest 200 is a sleeveless, full-torso garment covering the chest and abdomen of a user from about the collar/shoulder region to approximately the hip. The vest 200 includes a V-neck collar opening at the top that can mate with the neck and/or shoulder region of the transcranial device 11, creating a continuous therapeutic coverage zone from the scalp to the abdomen. The vest 200 includes shoulder straps 201 and a rear upper back strap 221 to secure the vest 200 to the user's body. To ensure comfort and suitability, the vest 200 is produced in a range of sizes, and includes a hook-and-loop closure 220 to adjustably accommodate varying chest and torso circumferences of individual users.

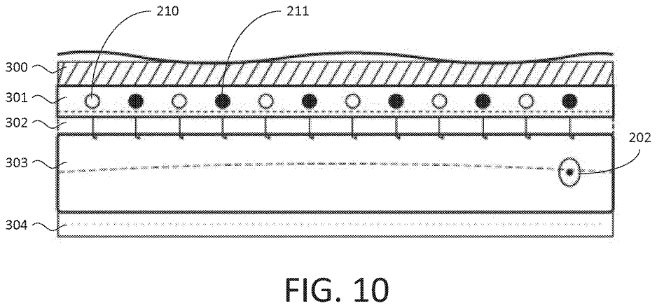

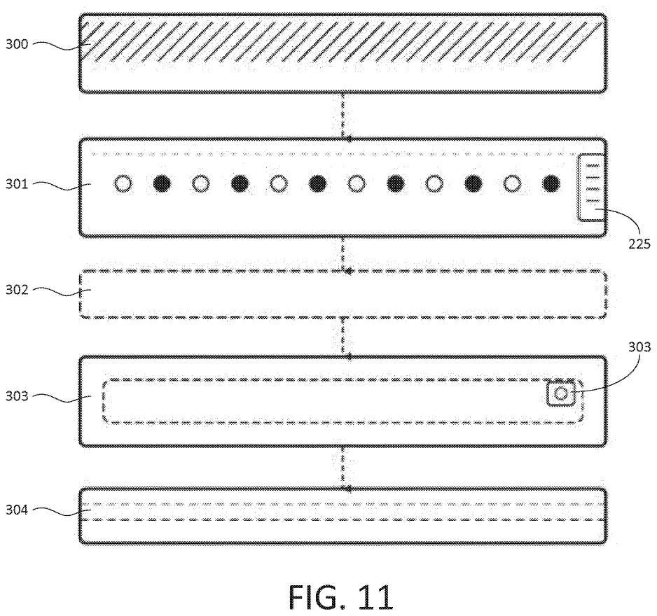

The vest 200 can be constructed from medical-grade materials (e.g., neoprene or an antimicrobial stretch knit base material). The vest 200 can be made of multiple layers. By non-limiting example, the vest 200 has five layers (FIGS. 10 and 11). The first layer 300 (the innermost layer) is a soft inner lining that can be antimicrobial. It can sit directly on a user's skin or thin clothing. The second layer 301 is a flexible PCB that includes an arrangement of photoemitters, such as LED emitters 210 and laser diodes 211. The third layer 302 is a silicone overmold that prevents ingress of dust and/or water. The third layer 302 can have an IP67 rating. The fourth layer 303 is a pneumatic bladder that can be filled with air to create a compressive force on the user. The fourth layer 303 can be made of thermoplastic polyurethane. The fifth layer 304 (the outermost layer) is a base fabric that can be made of neoprene or an antimicrobial stretch knit material.

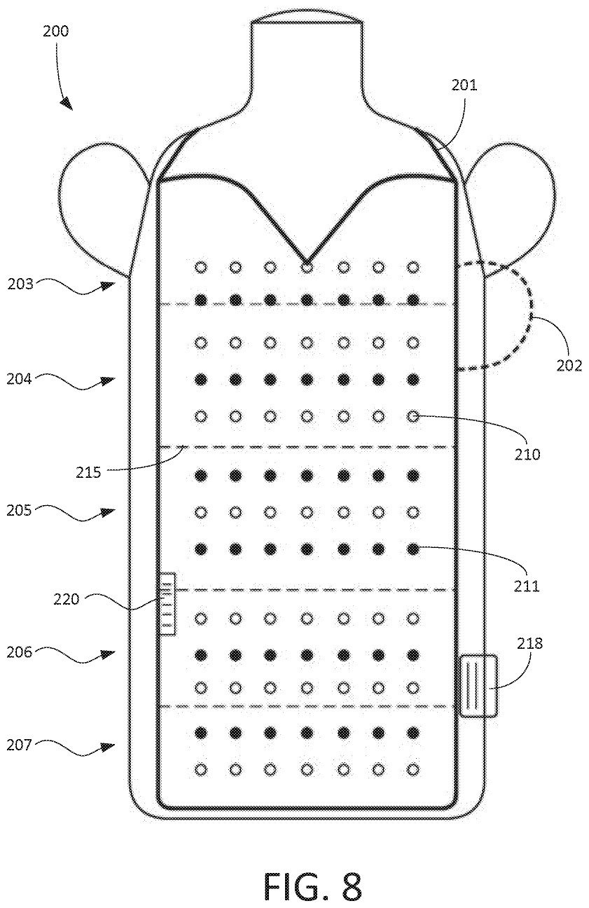

The vest 200 can be outfitted with a plurality of independent pneumatic compression cells. In the exemplary embodiment shown in FIG. 8, the vest 200 includes five independent pneumatic compression cells. The pneumatic compression cells are structured to encompass five horizontal zones across the vest 200. The first cell 203 is disposed at a collar and/or neck zone of the vest 200. The second cell 204 is disposed at an upper chest zone of the vest 200.

The third cell 205 is disposed at a mid-chest zone of the vest 200. The fourth cell 206 is disposed at a lower chest zone of the vest 200. The fifth cell 207 is disposed at an abdomen zone of the vest 200. Cells are separated from adjacent cells by a pneumatic cell boundary 215.

The pneumatic compressions cells are each fluidly connected to multiple air supply and return tubes 202. The air supply and return tubes 202 are fluidly linked to a solenoid valve assembly 250. The solenoid valve assembly 250 operates under control signals PneuCtrl generated by a solenoid driver 244. The solenoid valve assembly 250 is fluidly linked to an air pump 252 (e.g., a brushless DC motor pump) operated by a motor driver 246. Both the air pump 252 and the motor driver 246 can be situated within an external enclosure.

Further detailing these connections: an air supply line fluidly links the air pump 252 to the solenoid valve assembly 250. A return air supply line fluidly connects the solenoid valve assembly 250 to the air pump 252. A pressure supply line fluidly links the solenoid valve assembly 250 to an sensor 254. The air pressure sensor 254 can be located within the external enclosure. The air pressure sensor 254 is vital for detecting the air pressure within the pneumatic compression cells. Together, these components orchestrate the vasopneumatic compression on the user.

As described above, the vest 200 includes a second layer 301 with a flexible PCB that can have a plurality of LED emitters 210 and a plurality of laser diodes 211. The flexible PCB is laminated to the vest interior. In exemplary embodiments, there are up to 1,000 LED emitters 210 and up to 2,000 laser diodes 211. The LED emitters 210 and laser diodes 211 can emit light at a variety of wavelengths, thus allowing for various depths of light penetration into the user's body. Three predominant light wavelengths are used: a 660 nm wavelength that can penetrate 2-5 mm into the user's skin to incite superficial lymphatic stimulation; a 850 nm wavelength that can penetrate 5-10 mm into the user's skin to stimulate thoracic lymph node chains; and a 1064 nm wavelength that can penetrate 10-15 mm into the user's skin to stimulate deep substernal lymphatics. All above-described wavelengths promote mitochondrial cytochrome c oxidase activation, which increase ATP production, reduces reactive oxidative stress, activates anti-inflammatory cascades, and improves mitochondrial function and cellular metabolism across thoracic and abdominal tissue beds.

The LED emitters 210 and laser diodes 211 are oriented on the vest 200 so that there are three zones that each emit a primary wavelength of light into the user. It should be noted that every zone includes a mixture of wavelengths, but each zone has a primary wavelength emphasis. The first zone is the upper chest zone, which includes the second cell 204 and third cell 205, and primarily houses the 660 nm LED emitters 210 and laser diodes 211. The second zone is the mid-chest zone, which includes the third cell 205 and the fourth cell 206, and primarily houses the 850 nm LED emitters 210 and laser diodes 211. The third zone is the lower chest/abdomen zone, which includes the fourth cell 206 and the fifth cell 207, and primarily houses the 1064 nm laser emitters 210.

The LED emitters 210 and laser diodes 211 are powered by a battery module 230. The battery module 230 also provides power to the solenoid driver 244 and the motor driver 246 used for the pneumatic compression cells. The battery module 230 connects to a battery management system 232. The battery management system 232 is configured to prevent overcharging, over-discharging, over-currenting, short-circuiting, battery cell imbalance, and thermal battery issues. The battery management system 232 feeds a power management unit 234. The power management unit 234 regulates separate power rails: a 12 V rail for laser diode drivers 242, a 5 V rail for LED drivers 240 and a microcontroller unit 236, and 12 V to 15 V for the motor driver 246.

The power management unit 234 connects to a microcontroller unit 236. The microcontroller unit 236 can be removably connected to the vest 200 by the multi-pin vest connector 218. The microcontroller unit serves as a central hub, generating four simultaneous control signal channels: a PWM signal to the LED drivers 240, a PWM signal to the laser diode drivers 242, a PneuCtrl signal to the solenoid driver 244, and a PumpStart signal to the motor driver 246. The PWM signal to the LED drivers 240 controls the intensity and pulsing frequency of the LED emitters 210. The PWM signal to the laser diode drivers 242 controls the intensity and pulsing frequency of the laser diodes 211. Power is supplied to the LED emitters 210 and laser diodes 211 in the vest 200 by an internal wiring channel 225. The PneuCtrl signal controls the pneumatic compression cell inflation sequence and pressure applied to the pneumatic compression cells. The PumpStart signal controls the air pump 252. The microcontroller unit 236 may turn the air pump 252 on or off depending on pressure measurements from the pressure sensor 254.

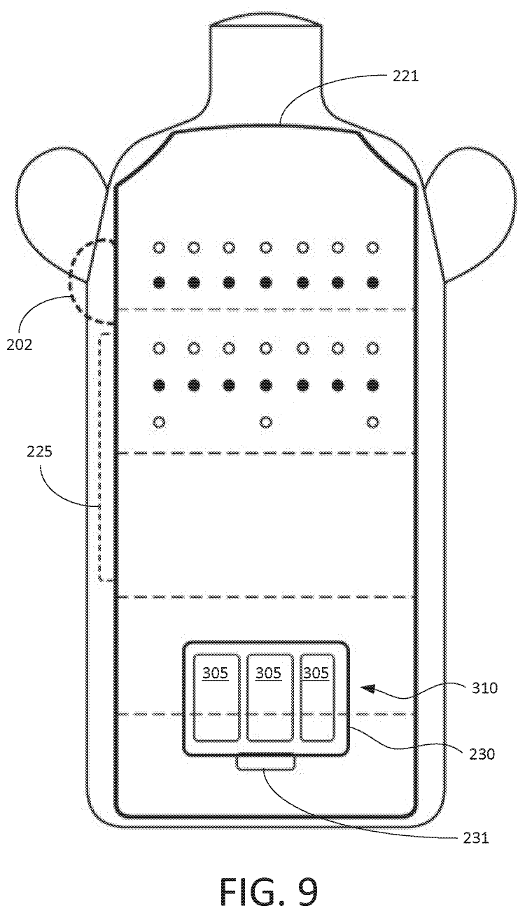

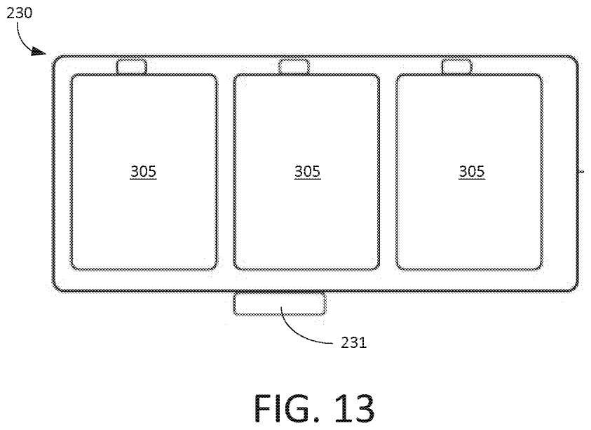

The battery module 230 includes at least one battery cell 305. In an exemplary embodiment, there are at least three battery cells 305 (FIG. 13). The battery cell 305 can be a rechargeable lithium polymer battery with about a 14.8 voltage in a 4S configuration, with a 10 Ah capacity, and connected in parallel. The battery module provides approximately 444 Wh in energy storage and supports at least three hours of continuous therapeutic operation of the vest 200. The battery module 230 includes a charge port 231 for recharging the battery module 230. The battery module 230 can be charged while the vest 200 is in use, or when the vest 200 is not being used. In exemplary embodiments the battery module 230 is integrated into the vest 200 and disposed on the rear of the vest in battery module pouch 310 (FIG. 9). In some embodiments the battery module 230 is contained within the external enclosure. The external enclosure can be clipped onto the user (e.g., by the user's clothing) or the external enclosure can be placed on a surface during treatment sessions. In other embodiments, the vest 200 also contains a pocket in which to place the external enclosure.

During treatment the vest 200 provides vasopneumatic photobiomodulation treatment to the user. Treatment is typically administered once daily, for a duration of 30 minutes per session. The treatment works in conjunction with the above-described treatment by the transcranial device 11 and the intraoral device 60 to remove toxins (e.g., hyperphosphorylated tau and amyloid beta) from the CNS. The vest 200 extends the treatment described above by adding thoracic and abdominal coverage. Complementing the use of the transcranial 60 and intraoral 11 devices, the vest 200 removes toxins upward through the thoracic duct. In combination, the transcranial device 60, intraoral device 11, and vest 200 provide coordinated pressure and photobiomodulation treatment along the full lymphatic drainage pathway.

The vest 200 accomplishes lymphatic drainage by compression of the pneumatic compression cells: the pneumatic compression cells are configured to inflate and deflate in a sequential manner. In an exemplary embodiment, upon activation, the vest 200 delivers a sequence of preset mmHg compression to the user's torso. The first cell 203 (also identified as the superior cell) receives the highest compression, and each subsequent cell receives a lower compression, at least 1 mmHg less than the preceding cell, with the fifth cell 207 (also identified as the inferior cell) receiving the lowest pressure. By non-limiting example, if the first cell 203 receives a compression of 10 mmHg, then the second cell 204 receives a compression of 9 mmHg, the third cell 205 receives a compression of 8 mmHg, the fourth cell 206 receives a compression of 7 mmHg, and the fifth cell 207 receives a compression of 6 mmHg. This creates a superior-to-inferior pressure gradient. The microcontroller unit 236 controls the sequence of compression amongst the pneumatic compression cells. In an exemplary embodiment, the microcontroller unit 236 causes the pneumatic compression cells to compress sequentially from the fifth cell 207 (the inferior cell) to the first cell 203 (the superior cell), thereby draining the majority of lymph upwards, towards the left subclavian vein.

In conjunction with the lymphatic drainage from the pneumatic compression cells, the vest 200 simultaneously provides photobiomodulation treatment through the LED emitters 210 and the laser diodes 211. Photobiomodulation occurs via the light emitted at specific wavelengths. The 660 nm light (primarily emitted by the second 204 and third 105 cells) stimulates dermal lymphatic capillaries overlying thoracic lymph nodes, reduces inflammatory cytokine production in skin and subcutaneous tissues, and improves capillary permeability. The 850 nm light (primarily emitted by the third cell 205 and the fourth cell 206) reaches thoracic lymph node chains, intercostal muscles, and anterior thoracic wall vasculature; stimulates lymphatic vessel smooth muscle contractility; upregulates aquaporin-4 water channel expression on astrocyte endfeet, thus improving CSF-ISF exchange (e.g., glymphatic throughput). The 1064 nm light (primarily emitted by the fourth cell 206 and the fifth cell 207) reaches the substernal lymph nodes, the internal mammary lymph node chain, and the central thoracic lymphatic trunks directly.

The photobiomodulation treatment can be offered in three different modes: 10 Hz mode, 40 Hz mode, and a continuous mode (also referred to as 0 Hz mode). In 10 Hz mode, light is emitted in a pulsing frequency from the LED emitters 210 and laser diodes 211 at a frequency of about 10 Hz. The 10 Hz frequency corresponds to alpha-band stimulation associated with parasympathetic activation and lymphatic motility. In 40 Hz mode, light is emitted in a pulsing frequency from the LED emitters 210 and laser diodes 211 at a frequency of about 40 Hz. The 40 Hz frequency corresponds to the gamma brainwave frequency range that drives glymphatic clearance and reduces amyloid-beta accumulation. The continuous mode causes light to be continuously emitted without any pulsation.

Vasopneumatic photobiomodulation treatment is controlled by the microcontroller unit 236. The microcontroller unit 236 may include predetermined sequences that provide a preset vasopneumatic photobiomodulation treatment. In some embodiments these instructions can be installed into the microcontroller unit via a USB interface, thereby allowing data transmission via a USB port with an external device. This interface provides a path for programming the microcontroller unit 236 or collecting data from the microcontroller unit 236. In other embodiments the microcontroller is configured to wirelessly connect with an external device, such as a cell phone or a computer, to program the microcontroller unit 236 provide a given treatment sequence. In other embodiments, the vest 200 includes a touch screen, which can be mounted on the vest 200 itself or on the external enclosure. The touch screen is electrically connected to the microcontroller unit 236. Under the control of the microcontroller unit 236, the touch screen displays operational options to the user and accepts input commands.

The effectiveness of these devices can be evaluated by comparing baseline blood work and follow-up measures after a set treatment period. For instance, blood tests for hyperphosphorylated tau, amyloid beta-40, (Aβ40, Aβ42), Apolipoprotein E prototype, and the Amyloid Probability Score (APS, calculated from plasma Aβ42/40 ratios) can be used to track the removal of Aβ from the CNS.

While this novel treatment strategy has been detailed in the context of mitigating cognitive impairment due to Alzheimer's disease, those of skill in the art will appreciate the potential applicability of the system and method to a broader spectrum of brain disorders. The transcranial device 11 and the intraoral device 60 are specifically designed to stimulate the glymphatic system and facilitate the clearance of macromolecules, including amyloid-beta (AB) and tau proteins, from the central nervous system (CNS). As these proteins are implicated in various neurodegenerative conditions beyond Alzheimer's disease, the use of this treatment regimen can be extended to these other brain disorders as well.

Indeed, many neurological conditions may be characterized by the accumulation of abnormal proteins in the CNS. For instance, Parkinson's disease may involve the buildup of alpha-synuclein protein in the brain, while Huntington's disease may result from an excess of the Huntingtin protein. Similarly, Amyotrophic lateral sclerosis (ALS) and Frontotemporal dementia (FTD) may be associated with the accumulation of TAR DNA-binding protein 43 (TDP-43).

In such conditions, there is a shared underlying theme of protein accumulation and impaired clearance, leading to cellular dysfunction and, eventually, neurological symptoms. By enhancing glymphatic and lymphatic flow within the CNS through the combined action of the transcranial device 11 and the intraoral device 60, it may be possible to increase the clearance of these proteins, potentially alleviating symptoms and slowing disease progression.

Therefore, the use of this innovative treatment strategy is not restricted to the treatment of Alzheimer's disease. Its potential extends to any neurological condition where the buildup of abnormal proteins contributes to disease pathology. It is a universal therapeutic approach that may play a significant role in combating a range of debilitating brain disorders associated with protein accumulation in the CNS.

The systems and devices described herein are configured to apply mechanical and photonic stimuli that may influence fluid movement within lymphatic and glymphatic pathways. References to physiological mechanisms or biological processes are provided for explanatory purposes and do not require that such mechanisms occur in order for the disclosed systems to operate as claimed.

If the specification or claims refer to “an additional” element, that does not preclude there being more than one of the additional elements.

It is to be understood that where the specification states that a component, feature, structure, or characteristic “may”, “might”, “can” or “could” be included, that particular component, feature, structure, or characteristic is not required to be included.

The description of the invention is intended to be read in connection with the accompanying drawings, which are to be considered part of the entire written description of this invention. In the description, relative terms such as “front,” “rear,” “lower,” “upper,” “horizontal,” “vertical,” “inward,” “outward,” “up,” “down,” “top” and “bottom” as well as derivatives thereof (e.g., “horizontally,” “downwardly,” “upwardly” etc.) should be construed to refer to the orientation as then described or as shown in the drawings under discussion. These relative terms are for convenience of description and do not require that the device be constructed or the method to be operated in a particular orientation. Terms, such as “connected,” “connecting,” “attached,” “attaching,” “join” and “joining” are used interchangeably and refer to one structure or surface being secured to another structure or surface or integrally fabricated in one piece.

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be #10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

Thus, the invention is well adapted to carry out the objects and attain the ends and advantages mentioned above as well as those inherent therein. While the inventive concept has been described and illustrated herein by reference to certain illustrative embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A system for treating neurodegenerative diseases involving accumulation of abnormal proteins in the central nervous system (CNS), the system comprising a vest, wherein the vest comprises a vasopneumatic compression system and a photobiomodulation system, the vest configured to exert vasopneumatic compression and photobiomodulation on a user's torso and abdomen to stimulate the user's lymphatic system and enhance the flow of lymphatic fluid towards the user's subclavian lymphatic pathway.

2. The system of claim 1, wherein the vest further comprises:
a microcontroller unit that adjusts the vasopneumatic compression and photobiomodulation treatment;
a LED driver connected to a plurality of LED emitters;
a laser driver connected to a plurality of laser diodes;
a solenoid driver connected to a solenoid valve, wherein the solenoid valve comprises a plurality of valve outlets; and
a motor driver connected to an air pump;
wherein the LED driver, laser driver, solenoid driver, and motor driver are connected to the microcontroller.

3. The system of claim 2, wherein the vest further comprises:
a plurality of pneumatic bladders, wherein the pneumatic bladders are inflated and deflated by the air pump;
a plurality of vest layers, wherein the plurality of vest layers comprises at least the pneumatic bladder and a flexible PCB, and wherein the flexible PCB comprises the plurality of LED emitters and the plurality of laser diodes.

4. The system of claim 3, wherein the vest further comprises an air pressure sensor that measures pressure in the plurality of pneumatic bladders and is connected to the microcontroller unit and the pressure sensor.

5. The system of claim 4, wherein the vest:
exerts vasopneumatic compression by sequentially inflating and deflating the plurality of pneumatic bladders within the vest, wherein the pneumatic bladders are sequentially inflated from an inferior cell to a superior cell and each cell from the inferior to the superior cell decreases in compression; and
provides photobiomodulation by the plurality of LED emitters and laser diodes, wherein:
the plurality of LED emitters emit a wavelength of light selected from the group consisting of 660 nm and 850 nm;
the plurality of laser diodes emit a wavelength of light selected from the group consisting of 660 nm, 850 nm, and 1064 nm; and
the plurality of LED emitters and the plurality of laser diodes pulse emitted light at a frequency selected from the group consisting of 10 Hz, 40 Hz, and 0 Hz.

6. The system of claim 5, wherein the vest further comprises:
a power management unit;
a battery management system; and
a battery module, wherein the battery module provides power to the vest.

7. The system of claim 6, further comprising an external enclosure, wherein the solenoid driver, the solenoid valve, the motor driver, the air pump, the battery module, the battery management system, the power management unit, and the microcontroller unit are disposed within the external enclosure.

8. A system for treating neurodegenerative diseases involving accumulation of abnormal proteins in the central nervous system (CNS), the system comprising:
an intraoral device designed to fit hard and soft palates of a user, the intraoral device comprises a vibration mechanism and a set of photobiomodulation elements and is configured to apply photobiomodulation and vibration to specific points on the hard and soft palate of the user to stimulate the user's glymphatic system and decongest tau and amyloid proteins and toxins;
a transcranial device comprising a head and neck garment having integrated vasopneumatic compression elements and photobiomodulation elements, the transcranial device configured to exert vasopneumatic compression and photobiomodulation on the user's head and neck to stimulate the user's lymphatic system and enhance the flow of lymphatic fluid towards the user's subclavian lymphatic pathway; and
a vest, wherein the vest comprises a vasopneumatic compression system and a photobiomodulation system, the vest configured to exert vasopneumatic compression and photobiomodulation on a user's torso to stimulate the user's lymphatic system and enhance the flow of lymphatic fluid towards the user's subclavian lymphatic pathway.

9. The system of claim 8, wherein the vibration mechanism of the intraoral device includes a motor driver that drives a linear vibration motor, and wherein the set of photobiomodulation elements of the intraoral device includes an LED driver that drives a plurality of visible red and NIR LEDs.

10. The system of claim 8, wherein the transcranial device includes a garment configured to exert vasopneumatic compression and photobiomodulation on the user's head and neck region, and wherein the transcranial device further includes a control unit that adjusts the vasopneumatic compression and photobiomodulation intensity in response to pre-set treatment protocols.

11. The system of claim 8, wherein the vibration mechanism and the set of photobiomodulation elements of the intraoral device are configured to apply vibration and photobiomodulation to a plurality of specific points on the hard and soft palate.

12. The system of claim 8, wherein the vibration mechanism and the set of photobiomodulation elements of the intraoral device are configured to apply vibration and photobiomodulation to seventeen specific points on the hard and soft palate.

13. The system of claim 8, wherein the abnormal proteins include tau and amyloid-beta proteins.

14. The system of claim 8, wherein the vest comprises a microcontroller unit that adjusts the vasopneumatic compression and photobiomodulation intensity to pre-set treatment protocols.

15. A method of treating neurodegenerative diseases involving accumulation of abnormal proteins in the central nervous system (CNS) of a user, the method comprising:
utilizing a vest to exert vasopneumatic compression and photobiomodulation on a user's torso to stimulate the user's lymphatic system and enhance the flow of lymphatic fluid toward the subclavian lymphatic pathway.

16. The method of claim 15, wherein the vest is utilized to apply vasopneumatic compression to propel lymph upward through a thoracic duct.

17. The method of claim 16, wherein the step of utilizing the vest further comprises the steps of:
creating a pressure gradient that increases from an inferior cell of the vest to a superior cell of the vest, wherein the pressure gradient is created from sequentially inflating a plurality of cells within the vest;
emitting light from the vest into the user, wherein
the light is emitted by a plurality of photoemitters;
the each photoemitter emits a wavelength of light selected from the group consisting of 660 nm, 850 nm, and 1064 nm; and
pulsing the emitted light, wherein each photoemitter pulses the wavelength of light at a frequency selected from the group consisting of 0 Hz, 10 Hz, and 40 Hz.

18. The method of claim 17, further comprising:
simultaneously with the utilization of the vest, utilizing an intraoral device to apply photobiomodulation and vibration to specific points on hard and soft palates of the user to stimulate the user's glymphatic system and decongest tau and amyloid proteins and toxins; and
simultaneously with the utilization of the vest and intraoral device, applying a transcranial device comprising a head and neck garment to exert vasopneumatic compression and photobiomodulation on the user's head and neck to stimulate the user's lymphatic system and enhance the flow of lymphatic fluid toward the subclavian lymphatic pathway.

19. The method of claim 18, wherein the abnormal proteins include hyperphosphorylated tau and amyloid-beta proteins.

20. The method of claim 19, wherein the neurodegenerative disease is selected from the group consisting of Alzheimer's disease, Parkinson's disease, Huntington's disease, and Amyotrophic lateral sclerosis (ALS).
